(19) United States
(12) Patent Application Publication
(10) Pub. No.: US 2012/0259302 A1
(43) Pub. Date: Oct. 11, 2012

(54) SURGICAL PRODUCT

(75) Inventors: Boun Chong Chaisumdet, Breakfast Point (AU); Ben S. Chen, Cecil Park (AU)

(73) Assignee: Multigate Medical Products Pty Limited

(21) Appl. No.: 13/500,060

(22) PCT Filed: Aug. 3, 2010
(86) PCT No.: PCT/AU2010/000974

§ 371 Date: Jun. 11, 2012

(30) Foreign Application Priority Data
Aug. 3, 2009 (AU) 2009903602
Dec. 8, 2009 (AU) 2009905964

(51) Int. Cl. A61F 13/53; B29C 47/00

(52) U.S. Cl. 604/367; 264/172.19

(57) ABSTRACT

A surgical product being a swab or sponge (10, 40), the surgical product including a nonwoven sheet (81, 42, 44) made up of multiple layers of a non-woven synthetic material and including a detectable element in the form of an X-ray- or radio-detectable element (50, 52, 60).

### FIELD OF INVENTION

This invention relates to a surgical product. More particularly, this invention relates to a gauze or swab. Still more particularly, this invention relates to an X-ray or radio detectable surgical gauze or swab, including abdominal sponges.

### BACKGROUND ART

The following references to and descriptions of prior proposals or products are not intended to be, and are not to be construed as, statements or admissions of common general knowledge in the art. In particular, the following prior art discussion does not in relate to what is commonly or well known by the person skilled in the art, but assists in the understanding of the inventive step of the present invention of which the identification of pertinent prior art proposals is but one part.

Surgical cloth materials for dressings, swabs, sponges and the like are traditionally made of woven cotton gauze. Natural fibre cloths comprising a high proportion of cotton are difficult to maintain as sterile and there are high production costs involved in the removal of impurities and contaminants, for example following bleaching.

It has been proposed to make swabs and sponges partly using synthetics such as polyester, and/or blends with wood pulp. Purely synthetic cloths may comprise a range of different types of polymers, copolymers and polymer blends. Suitable materials that may be utilized in medical cloth include alginate, cellulose, urethanes, hydro-gels, hydrocolloids and copolymers of acrylates and acrylamides, amongst many others.

Because X-ray Detectable Sponges and Swabs are primarily used inside body cavities during invasive surgical procedures to soak up blood, it is desirable that gauze, swabs and other medical cloths display properties of low and consistent bioburden and consistent weight. The former aids in maintaining the desired sterility assurance level as prescribed in ISO 11137; the latter helps health professionals in calculating blood loss from the patient.

Predominantly polypropylene-based products have been considered by some research to be unsuitable for surgical applications.

Polypropylene belongs to the family of polyolefins or polyalkenes and is a thermoplastic polymer resin of propylene made from petroleum. Polypropylene is moulded or extruded into many forms, including fibres for suture material, mesh and nonwoven sheets. Although nonwoven polypropylene mesh possesses attractive properties of high absorbency, good bacterial nonadherence, good pliability and moldability for use in surgical packing, the material has generally been considered irritating to touch and medical professionals may tend to avoid its use. Although nonwoven polypropylene materials have been used in the manufacture of products such as sterilisation wraps for sterilising instruments preparatory to surgery, they have not been thought of as suitable for use in the manufacture of surgical gauze or swabs.

Woven swabs and sponges can also suffer from the problem that loose threads, liable to separate from the main body of a swab, can cause contamination or infection. There is therefore a need to provide a product which does not shed loose threads.

Furthermore, a significant systemic problem, in terms of surgical procedure, is the problem of swabs being left in a patient's wound or body cavity following completion of a surgical procedure. Gauze swabs, saturated with fluids such as blood or mucus, are notoriously difficult to distinguish from surrounding human tissue and body fluids, due to the similar consistency and appearance of the used gauze to the surrounding tissue. To prevent medical materials and cloth from being inadvertently left in a patient's body cavity following suturing, a common surgical protocol involves specifically counting each swab on entry and removal from the body cavity or wound. However, despite the best efforts of medical professionals, the inadvertent failure to remove medical materials from a wound or other body cavity continues to be a problem, that may lead to septicaemia and, occasionally, death.

Accordingly, the need persists for effective means of detecting surgical products that are inadvertently left in the body of a patient and preventing this occurrence in the first instance. Therefore, sterile X-ray detectable sponges and swabs are typically supplied in packs of 5 having a determinable weight. Having consistent weight is highly desirable in the manufacturing process because the count of items entering a patient's body (during an operating procedure) is validated by weight of the pack.

An object of the present invention is to ameliorate the aforementioned disadvantages of the prior art or to at least provide a useful alternative thereto.

### STATEMENT OF INVENTION

Accordingly, there is provided a surgical product being a swab or sponge, the surgical product including a single nonwoven sheet made up of multiple layers of a non-woven synthetic material and including an X-ray- or radio-detectable element.

Preferably, there is provided a surgical product and a method of making same comprising the combination of the overlaying of multiple layers of non-woven material, preferably alternately oriented in machine and cross directions, and having an X-ray or radio detectable element, preferably barium sulphate coated thread or ribbon, preferably aligned in both machine and cross directions, and preferably crossing along their respective lengths.

Preferably, the surgical product is wholly or predominantly made from wood pulp and polyester spun bonded layers of non-woven material, the single sheet folded over one or more times. For example, a 10×10 cm sheet may be folded once to form a rectangular swab and twice or more to form a sponge. In UK, Australia and in NZ, if the folded surgical product is also stitched together, it is generally referred to as a “sponge”, and if it is simply folded without stitching the folder layers, it is referred to as a swab. Typical swabs are 100 mm×100 mm and 100 mm×150 mm, 4-ply-16-ply. For example, a 100 mm×100 mm 4-ply swab is made from a 200 mm×200 mm sheet. A “sponge” is usually made from a 250 mm×250 mm sheet. The range of products that are used for packing include 125 mm×1 m rolls and may be referred to as “X-ray Detectable Packing Gauze”. Such packaging products may be provided in rolls having widths between 10 mm to 125 mm, and preferably widths of 12.5 mm to 100 mm, and in lengths of 1 m to 2 m.

Optionally, one or more sheets of the same or a different material may be sandwiched within the folded laminated sheet. Preferably, a single sheet of the same material is sandwiched within the folded sheet.

Preferably, the sheet is wholly or predominantly made from wood pulp/polyester fibres. The material constituting such products is generally characterised by good physical strength, a high body fluid absorption capacity and hypo-allergenic properties. The surgical cloth product also advantageously retains its integrity when wet so that loose fibres or particles thereof are not easily separated from the main body of the product when used as a swab or sponge or as packing/spacing material during a surgical procedure.

The surgical cloth product fibres may be impregnated with agents to improve and modify the properties of the material. Advantageously, however, and in a particularly preferred aspect of the invention, the surgical cloth product may be sterilised by ethylene oxide or irradiation after they are counted and packed.

The surgical cloth product may be made from one or more (bided sheets with X-ray detectable thread or tape embedded or attached by means of heat bonding. The X-ray detectable thread or tape is usually made from a barium sulphate and polymer composite. The thread or tape may be coloured a dark colour, such as blue or black, for identification against the typically light-coloured background (such as white) of the surgical cloth product. The folded sheets may be bonded one layer one to another. Bonding may be effected by arrangements of suitable means, such as ultrasonic bonding, bonding using heat, stitching or by the use of adhesives. The bonding may be achieved by continuous sealing. Alternatively, the bonding may be achieved by spot sealing. In its simplified form, surgical cloth product may simply be folded without any bonding or stitching.

The surgical product may come in a variety of sizes and shapes. Preferably, the surgical product is rectangular in shape. The layers of material may be joined one to another by stitching around the edges. In a particularly preferred embodiment, the material is a synthetic, non-woven polypropylene fibre material.

In a particularly preferred form of the invention, the surgical cloth product may be in the form of a sponge, in particular an abdominal sponge for assisting in the cleaning out of wounds and as packing against body tissues during a surgical procedure.

The surgical product may be provided in a range of sizes. The surgical product may be square shaped. Sizes may range from 10 cm square through to 50 cm square whereby different sizes suit different applications. The nonwoven sheet may be folded once and stitched or otherwise bonded as set out above. In one embodiment, the primary nonwoven sheet is folded about a secondary separate single sheet, stitched and then folded again, to form a sponge which may be unfolded to act as a swab, this giving versatility to the product.

The product may be stitched or otherwise bonded in any chosen pattern but preferably is stitched in a Z or X pattern or simply folded without any stitching or bonding.

The X-ray- or radio-detectable element may comprise an X-ray opaque material made from barium sulphate held together by an inert polymer. The X-ray opaque material may be in the form of a wire, thread, ribbon, scam or bead interposed across, through or between the spun bonded fibres, the laminate layers or the sheet layers. Preferably, the element is in the form of a thread that is hydroentangled with one or more of the spun bonded layers. Even more preferably, the element is a thread hydroentangled with a secondary separate single sheet which is then enclosed when the primary nonwoven sheet is folded about it.

The X-ray opaque material may alternatively be particulate. In the latter case, the X-ray opaque material may be sprayed onto the fibres or may be otherwise impregnated by emersion, by brush application, injection or spraying. Preferably, the X-ray opaque material is in the form of filament having sufficient flexibility to bend with the fibres during use.

The X-ray- or radio-detectable element is advantageously not susceptible to separation from the surgical cloth product in use, preferably by hydroentanglement with, or bonding to, the surgical cloth fibres.

The X-ray opaque material may comprise a range of suitable materials having large atomic particles adapted to display good X-ray-detectable properties. For example, barium salts such as barium sulphate are particularly preferred.

The X-ray detectable elements may be added by planting or spraying them into the fibre web or into the surface of the fibre web or by heating them into the surface of the surgical cloth product. The X-ray detectable element is preferably barium sulphate thread. The X-ray detectable element may optionally include barium phosphate. The barium phosphate component may be in the form of a coating applied to a synthetic thread, such as polypropylene extruded thread. The X-ray detectable element may be heated onto the surface of the non-woven cloth, for example by hot heating, hot pressing or ultrasonic wave treatment.

A sheet of the surgical cloth may comprise two, four or more layers. The surgical product may be in the form of an abdominal sponge. The abdominal sponge can be made with three layers of fabric. In such cases, the edges of the respective layers are preferably stitched or welded as described herein. The surgical product may be in the form of a swab. The swab may have the plan dimensions 100 mm×100 mm. The swab may be made by folding a 200 mm×200 mm fabric twice.

Each layer of non-woven web or fabric may be separately manufactured by an extrusion process in which fibres or filaments are deposited on a forming surface in a so-called Machine Direction (MD) corresponding to the direction of travel of the extrusion machine. Each layer is preferably made from non-woven spun bonded material. Cross fibres are simultaneously laid perpendicular to the MD in a cross-direction (CD). The strength of the resulting fabric or web layer in the MD is considerably stronger than in the CD. The present Applicants have found that by overlaying multiple layers of fabric in alternating perpendicular orientation, relative to one another, then the resulting multi-layered sheet is extremely strong in both MD and CD of the first layer. Preferably sheets of non-woven material comprise four or more layers of non-woven fabric alternatively layered in MD and CD directions. Preferably, the layers rut joined, preferably ultrasonically or by stitching, at the sheet's periphery to secure the layers in overlaid relationship relative to one another. The stitching or welding may be continuous, may be intermittent and spaced, or may be extended only along one or more edges and not other sides of the sheet.

Preferably the X-ray detectable element is in the form of a strip or ribbon oriented in the MD, although the X-ray detectable element may be alternatively or additionally oriented in the CD. By alternatively layering layers in the MD and CD, the X-ray elements cross paths. This arrangement provides an improved visual clue to users of the presence of the X-ray element, because of the distinguishable opaque line extending in two different directions. It also informs the user, notably theatre stair, of the MD of a particular layer and also improves the X-ray detectability of the product in the unfortunate event that it is inadvertently left in a patient post-surgery.

The radio-detectable element may be a passive inductor chip trapped in the tangled fibres of the surgical product. The inductor chip is thus detectable by an RF device that avoids the further use of X-ray and the deleterious affects such use may have on patients and medical staff. The inductor chip may comprise peripheral nodules, hooks, notches, apertures or other surface features with which the fibres of the surgical product material may mesh, for example by hydraulic entanglement.

Because the product of the present invention presents an external surface comprising non-woven synthetic material, the present invention provides a surgical product capable of having a lower bacterial content compared to cotton or other non-synthetics. The material is easier to be sterilised than prior art materials, contains less contaminants because there is no need to bleach the material compared to, for example, cotton gauze and the chosen materials are particularly suitable to manufacture in a closed and controlled environment. The non-woven synthetic materials do not need stitching like traditional gauze, although stitching may be used for aesthetic reasons and to give the product a better feel. Finally, the non-woven synthetic materials contemplated according to the present invention maintain good integrity so that loose threads, particles or fibres do not disassociate from the main body of the surgical product under normal use, therefore improves healing time for patients. The more consistent weight helps in mounting during manufacturing and also make blood loss calculation more accurate.

### BRIEF DESCRIPTION OF THE DRAWINGS

The invention may be better understood from the following non-limiting description of so preferred embodiments, in which:

FIG. 1 is a plan view of a surgical product according to a preferred embodiment of the invention;

FIGS. 2a-2f are schematic side sectional views of various embodiments made according to the invention;

FIG. 3 is a schematic side sectional view of a surgical product according to another embodiment of the invention;

FIG. 4 is a schematic plan view of another embodiment of the invention;

FIG. 5 is a schematic plan view of a portion of a layer of non-woven fabric according to another embodiment of the invention;

FIG. 6 is a schematic end view of a portion or a sheet made according to another embodiment of the invention; and

FIG. 7 is a schematic plan view according to another embodiment of the invention.

### DESCRIPTION OF THE DRAWINGS

Preferred features of the present invention will now be described with particular reference to the accompanying drawings. However, it is to be understood that the features illustrated in and described with reference to the drawings are not to be construed as limiting on the scope of the invention.

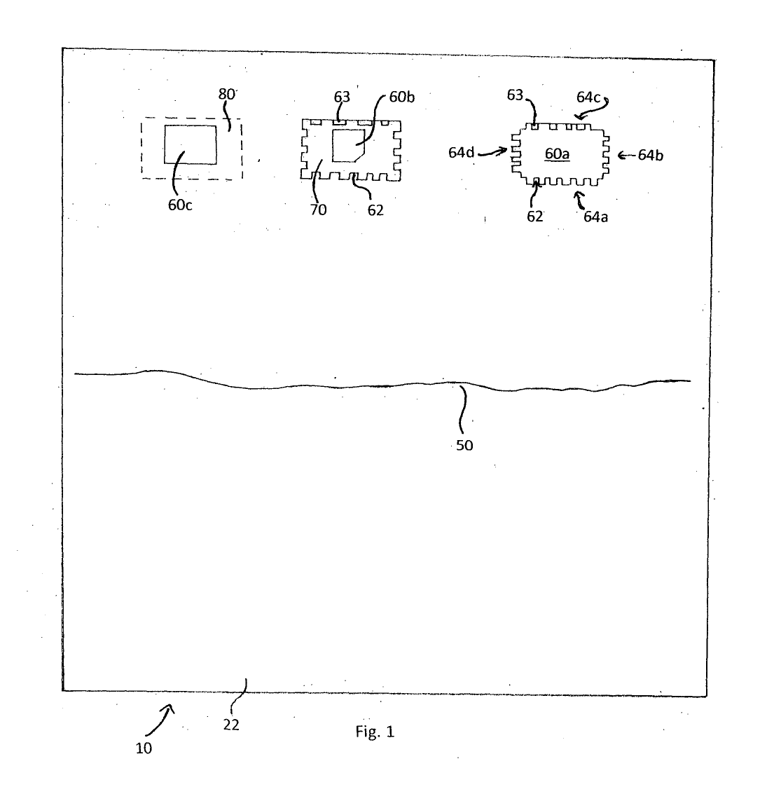

Referring to FIG. 1, there is shown a surgical product in the form of an abdominal sponge or swab 10 that is substantially square in shape and has the general dimensions of approximately 200 mm². The sponge or swab 10 according to the invention also comes in a range of other sizes, for example 100, 200, 300, 400, 500 mm², noting that larger or smaller sizes and shapes are also within the scope of the present invention. The surgical product 10 includes an X-ray detectable element 50 comprising barium sulphate thread interposed in the fibres 22 of the surgical product 10. The X-ray detectable thread 50 may be hydraulically entangled into the fibres 22 to achieve an effective bond of the non-woven fibres of the surgical product to encapsulate and capture the thread 50. The thread 50 may alternatively be threaded through the fibres 22 in a Z or double-pass X pattern.

In an alternative arrangement conveniently shown in FIG. 1, the X-ray detectable element 50 may be substituted with a passive RF inductor chip 60a. The chip 60a has peripheral notches 62 and/or holes 63 along at least one of its edges 64a and preferably all sides 64a-d. The nonwoven fibres 22 are hydro entangled with the notches 62 to non-removably secure the chip 60a in the swab 10.

In another arrangement, the inductor chip 60b may be unmodified from the standard planar plate type design having no specific edge or surface entangling features and may instead be trapped in a capsule 70 possessing the peripheral features 62, 63 suitable for entanglement with the fibres of the swab or sponge 10.

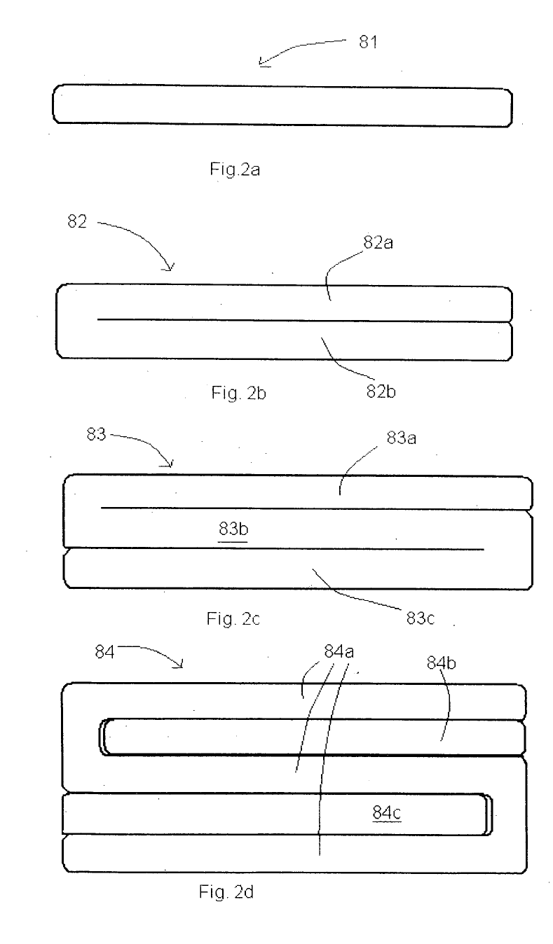

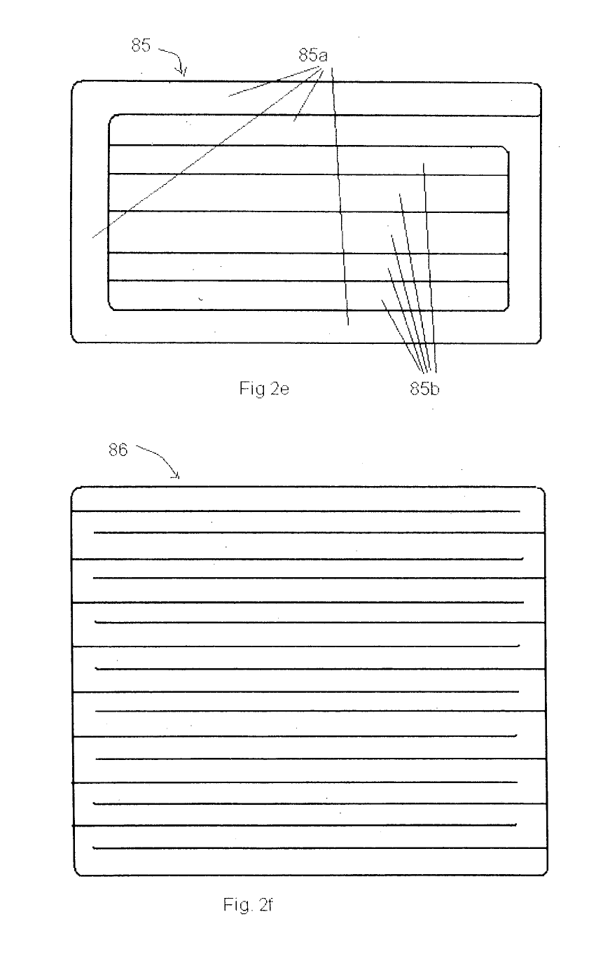

In still another arrangement, the RF chip 60c as shown in FIG. 1 may be trapped in a stitched pocket 80 between layers 82a, b (described below with reference to FIG. 2).

With reference to FIGS. 2 a-f, the sponge or swab 10 may be made from spun laced or bonded non-woven material comprising a cellulosic material combined with a medically inert synthetic polymer. In a particularly preferred form of the invention, the swab or sponge 10 is made from wood pulp and polyester spun laced or bonded to form single layer non-woven sheets. In the examples shown in FIGS. 2a-2f, the single sheet 81 may be provided as a single, unfolded sheet as shown in FIG. 2a, or may be folded over multiple times, for example to form a swab 820r sponge 83 as shown respectively in FIGS. 2b and 2c. Alternatively, as shown in FIGS. 2d and 2e, a single sheet, folded over may be combined with additional single sheet inserts as will be described in more detail below. Advantageously, the non-woven material of the swab or sponge 10, 81-86 has the desirable property of high friction between folded or inserted layers whereby the various layers do not naturally slip relative to one another. Furthermore, there is minor entanglement between the fibres 22 of adjacent layers whereby multiple layers tend to combine together to form a unitary product whose layers tend not to slip or separate one from the other.

In particular, in FIG. 2b there is shown a swab 82 comprising a single sheet folded over to form a pair of adjacent layers 82a, b lying in generally in parallel planes in a flattened, unused state. Stitching is not required due to the sufficient association of adjacent surface fibres of the respective layers 82a, b.

In FIG. 2c, a sponge 83 is provided. The sponge 83 is formed from a single sheet folded over twice to form three layers 83a-c that are folded in a Z pattern to sandwich middle layer 83b between outer layers 83a, c. The respective layers 83a-c tend not to disassociate from each other due to the slight entanglement of surface fibres 22 between the layers.

FIG. 2d shows a variation on the embodiment shown in FIG. 2c in the form of a sponge 84 comprising a single layer 84a folded over twice to form three layers spaced by a pair of spaced inserts 84b, c, being smaller sheets of the same or different material. In this regard, the ability to provide inserts 84b, c provides the opportunity to insert layers having different properties of; for example, compressibility, resiliency, absorbency, strength, flexibility, rigidity and the like. In any case, different sheets 84a, b, c optionally having different properties may still comprise a combination of cellulosic and synthetic polymer spun laced or woven into single sheets.

Referring to FIG. 2c, there is shown a sponge comprising a single sheet 85a wrapped around multiple adjacent inserts 85b. Again, stitching is not required.

With respect to FIG. 2f, the inventive product may be in the form of a sponge 86 comprising a single elongate sheet folded over many times on itself, for example, in a Z pattern as shown to achieve a highly absorbent sponge 86. Such a sponge is suitable for use in, for example, UK surgical procedures.

It is note worthy that the embodiment shown in FIG. 2b, namely the swab 82 comprises the single shoot 82a, b which is folded over by half along a line 12 to form a highly absorbent dual layer pad 10 capable of absorbing up to twelve times its own weight. The respective free ends and edges of the folded over layers of sheet 82 may optionally be stitched together by polypropylene thread 14. However, it is noted that such stitching is not necessary and may simply encourage usage due to its resemblance to prior art, stitched gauze products.

Accordingly, the double layer of sheet 82 provides a pair of layers 82a, b made of non woven wood based and surgical grade polymeric material that is highly absorbent and resistant to bacterial adherence, whilst maintaining good tactile and aesthetic properties, as well as the capacity for high absorption of bodily fluids.

The X-ray detection thread 50 is inculcated into the first spun bond layer 82a by hydraulic bonding subsequent to the formation of the single sheet of swab 82 and subsequent to the folding over of the single sheet and the optional stitching 14 together thereof.

Once the thread 50 is hydroentangled into the fibres 22, the swab, sponge or pad 81-86 is subjected to sterilising radiation or steaming in an autoclave to achieve sterilisation and they are then stacked and packaged in a plastic sealed envelope in a closed and sterile environment.

In use, the packaged product 10, 81-86 is removed under sterile conditions, for example in surgery, and utilized as a gauze, pad, sponge or swab during, for example, a surgical procedure.

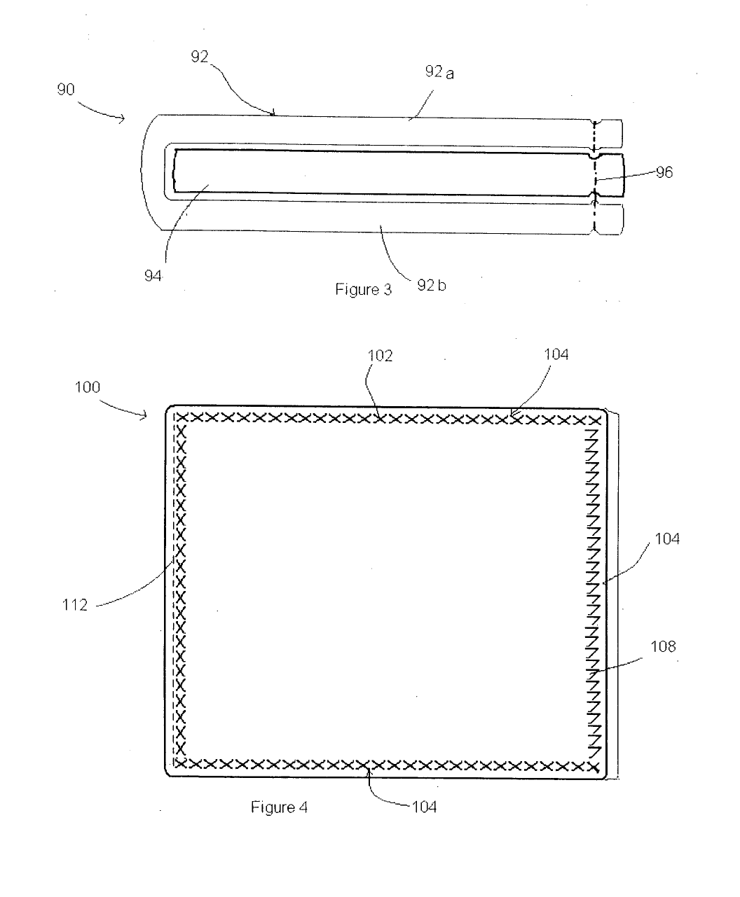

Turning to FIG. 3, in another embodiment of the invention the surgical product is in the form of a non-woven fibre sheet 92 folded about another single core sheet 94, to form a triple-layered product 90 suitable for use as a sponge. The respective pair of spaced layers 92a,b of the outer folded sheet 92 are stitched together by stitching 96 to envelop the inner single and non-folded sheet 94.

In still another embodiment shown in FIG. 4, the surgical product 100 may be stitched around by stitching 102 its borders 104, even along a fold 112. Preferably, the stitching is done in an X-pattern 106 or alternatively in a Z-pattern 108 for compliance purposes, that is to encourage usage of the product by ensuring that it looks and feels similar to prior art stitched products.

Moreover, although stitching may be optionally provided for primarily aesthetic, rather than functional purposes, it will be appreciated that stitching may also achieve functional differences, such as improved strength and resistance to disassociation of loosely adhered-to layers.

In FIG. 5 there is shown a single non-woven fibre sheet 30 comprising extruded polymeric material in which the dominant alignment of fibres is in the Machine Direction (MD) and having weaker cross fibres laid in the Cross Direction (CD). MD is the direction in which the extruding machine travels and produces the strongest and thickest fibres in the MD as represented by thicker fibres 24 in this direction, relative to the thin-fibres 26 in the CD. Inlaid and/or entangled in the sheet 30 in the MD is a strip of X-ray detectable ribbon, wire or strip 52 aligned in the MD.

As a skilled person will appreciate, the sheet 30 is much stronger in the MD, relative to the CD. However, it is customary to overlay multiple sheets in the same alignment direction, that is all overlaid sheets are aligned in, for example, the MD.

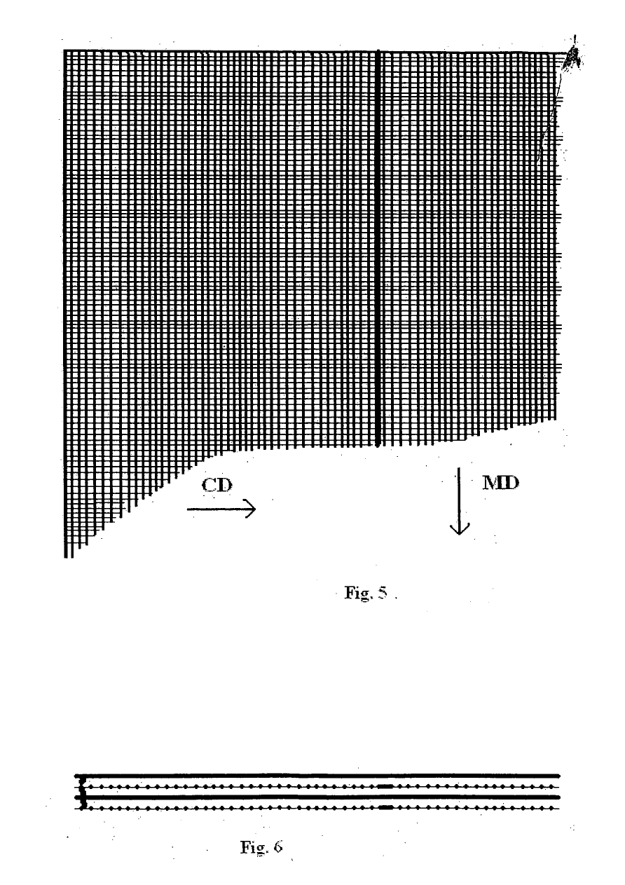

Counter to the traditional approach of aligning multiple overlaid sheets in the same direction, as shown in FIG. 6 multiple sheets are alternately aligned in MD and CD. The sponge or swab 40 comprises first and third sheets 42a, b aligned in a first direction D and second and fourth sheets 44a, b are aligned in a direction perpendicular to D. Of course, more than four layers 42a, b, 44a, b may be combined in overlaid relationship or pairs or triplets of adjacent layers may be aligned in parallel with subsequent groups of layers alternatingly aligned perpendicular to the first group of layers.

In the embodiment shown in FIG. 6, the surgical product 40 possesses good strength in both direction D and in the direction perpendicular to D. Moreover, the alternate alignment of adjacent sheets 42a, b, 44a, b provides corresponding cross alignment of respective X-ray detectable elements 52 in adjacent sheets 42a, b, 44a, b. This has the an advantage of making the presence of the X-ray detectable element 52 more visible to the naked eye of users, such as theatre staff, and also improves the detectability of the X-ray elements 52 as a combination by their extension along perpendicular axis, thereby reducing the possibility of a false negative reading.

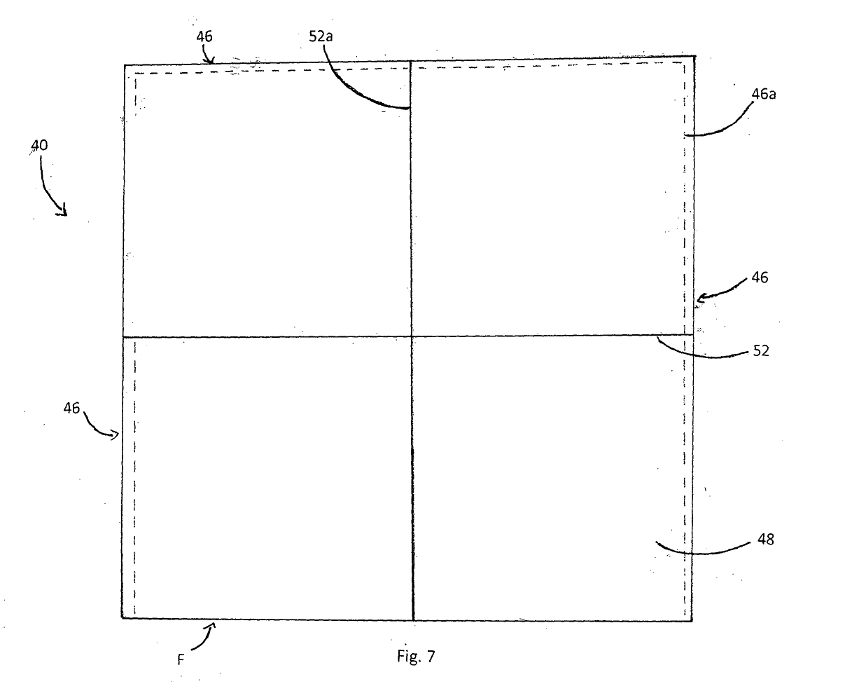

As shown in FIG. 7, the surgical product 40 may comprise a single sheet folded over along line F and stitched at its free peripheral edges 46 by either traditional stitching or by ultrasonic welding 46a as previously described. The surgical product 40 is a composite of multiple folded sheets. A first sheet 48 comprises a first X-ray detectable thread or element 52a aligned in a first direction (corresponding to the MD of the first sheet 48). A second sheet (obscured) is aligned perpendicular to the first sheet 48, whereby the MD of the second sheet is perpendicular to the MD of the first sheet 48 and the X-ray detectable thread 52a of the first sheet is also perpendicular to the X-ray detectable element 52b of the second sheet. The resulting surgical product 40 is equally strong and resistant to tearing or other loss of integrity in both the MD direction of the first sheet 48 and in the CD direction perpendicular to the MD, corresponding to the MD of the second sheet.

Not all layers of the surgical product 40 may include an X-ray detectable element 50,52. One, two or more of a multiple of layers may include the X-ray detectable element 50,52.

Throughout the specification and claims the word “comprise” and its derivatives are intended to have an inclusive rather than exclusive meaning unless the contrary is expressly stated or the context requires otherwise. That is, the word “comprise” and its derivatives will be taken to indicate the inclusion of not only the listed components, steps or features that it directly references, but also other components, steps or features not specifically listed, unless the contrary is expressly stated or the context requires otherwise.

Orientational terms used in the specification and claims such as vertical, horizontal, top, bottom, upper and lower are to be interpreted as relational and are based on the premise that the component, item, article, apparatus, device or instrument will usually be considered in a particular orientation, typically with the first half of layer 22, or the spaced layer 92a, uppermost.

It will be appreciated by those skilled in the art that many modifications and variations may be made to the methods of the invention described herein without departing from the spirit and scope of the invention.

1. A surgical product being a swab or sponge, the surgical product comprising:
a nonwoven sheet made up of multiple layers of a non-woven synthetic material and comprising a detectable element in the form of an X-ray- or radio-detectable element,
wherein the sheet comprises outer layers of spun bonded polypropylene fiber web and the internal layers comprise melt blown polypropylene fiber web and the detectable element is hydraulically entangled in one of the outer spun bonded layers.

2. The surgical product according to claim 1, wherein the detectable element comprises X-ray absorbent material.

3. A surgical product according to claim 2, wherein the X-ray absorbent material is a threaded element.

4. The surgical product according to claim 2, wherein the X-ray absorbent material comprises a polymeric thread coated with a barium salt.

5. The surgical product according to claim 1, wherein the detectable element is oriented in a machine direction corresponding to the direction of travel of an associated extrusion machine.

6. The surgical product according to claim 1, wherein at least one of the layers of the non-woven sheet is separately manufactured by an extrusion process in which fibers or filaments are deposited on a forming surface in a machine direction corresponding to the direction of travel of an associated extrusion machine.

7. The surgical product according to claim 6, wherein at least one of the layers of the non-woven sheet is separately manufactured by an extrusion process in which fibers or filaments are deposited on a forming surface in a machine direction corresponding to the direction of travel of the extrusion machine and wherein at least one of the layers of the non-woven sheet is made from non-woven spun bonded material.

8. The surgical product according to claim 2, wherein at least one layer of the non-woven sheet comprises cross fibers that are simultaneously laid perpendicular to the machine direction in a cross-direction.

9. A surgical product according to claim 8, wherein the strength of the layer in the machine direction is considerably stronger than in the cross direction.

10. A surgical product according to claim 8, wherein the multiple layers are overlaid in alternating perpendicular orientation, relative to one another, resulting in the multi-layered sheet being extremely strong in both the machine direction of a first one of the layers and in the cross direction of the first layer.

11. A surgical product according to claim 8, wherein the non-woven sheet comprises four or more layers of non-woven fabric alternatingly layered in the machine direction and in the cross direction.

12. A surgical product according to claim 8, wherein the multiple layers are joined ultrasonically or by stitching, at the sheet's periphery to secure the layers in overlaid relationship relative to one another.

13. A surgical product according to claim 8, wherein the X-ray detectable element comprises a first strip or ribbon oriented in the machine direction and a second strip or ribbon oriented in the cross direction so that the first and second X-ray detectable elements cross paths.

14. The surgical product according to claim 1, wherein the radio-detectable element is a passive inductor chip trapped in the tangled fibers of the surgical product.

15. The surgical product of claim 2, wherein the detectable element is oriented in a machine direction corresponding to the direction of travel of an associated extrusion machine.

16. The surgical product of claim 2, wherein at least one of the layers of the non-woven sheet is separately manufactured by an extrusion process in which fibers or filaments are deposited on a forming surface in a machine direction corresponding to the direction of travel of an associated extrusion machine.

17. A swab or sponge comprising:
a nonwoven sheet comprising multiple layers of a non-woven synthetic material and comprising a detectable element in the form of an X-ray- or radio-detectable element,
wherein the nonwoven sheet comprises:
outer layers of spun bonded polypropylene fiber web, and
internal layers comprising melt blown polypropylene fiber web, and
wherein the detectable element is hydraulically entangled in one of the outer layers, said detectable element being oriented in a machine direction corresponding to the direction of travel of an associated extrusion machine.

18. A swab or sponge comprising:
a nonwoven sheet comprising multiple layers of a non-woven synthetic material and comprising a detectable element in the form of an X-ray- or radio-detectable element, wherein the multiple layers are joined ultrasonically or by stitching, at the nonwoven sheet's periphery to secure the layers in overlaid relationship relative to one another, the nonwoven sheet comprising:
outer layers of spun bonded polypropylene fiber web, and
internal layers comprising melt blown polypropylene fiber web,
wherein at least one layer of the non-woven sheet comprises cross fibers that are simultaneously laid perpendicular to the machine direction in a cross-direction, and
wherein the detectable element comprises X-ray absorbent material hydraulically entangled in one of the outer spun bonded layers, said detectable element being oriented in a machine direction corresponding to the direction of travel of an associated extrusion machine.
